(19) United States
(12) Patent Application Publication
(10) Pub. No.: US 2017/0330480 A1
(43) Pub. Date: Nov. 16, 2017

(54) PREVENTION OF THE WRINKLING AND EXPRESSION OF HUMAN SKIN

(71) Applicants: Michelle L. Lubin, Princeton, NJ (US); Keith J. Hanna, Bronxville, NY (US)

(72) Inventors: Michelle L. Lubin, Princeton, NJ (US); Keith J. Hanna, Bronxville, NY (US)

(21) Appl. No.: 15/594,007

(22) Filed: May 12, 2017

Related U.S. Application Data
(60) Provisional application No. 62/336,728, filed on May 15, 2016.

(51) Int. Cl. G09B 19/00; A61M 35/00

(52) U.S. Cl. CPC G09B 19/00; A61M 35/00; A61M 2210/0606

(57) ABSTRACT

In some aspects, the present disclosure relates to methods for treating, preventing, minimizing, and/or diminishing signs of aging in the skin, such as facial wrinkles, and for training a user's face to favor some configurations or expressions over others. A gel or other viscous liquid with specific properties may be applied to areas on the face or body, that may change state to a thin, adhesive semi-elastic transparent film that is left on human skin over a period of time. The properties of the gel and the method for its application on the skin are carefully configured to for instance optimize the sensation (or force) feedback to the user in response to the user's conscious and sub-conscious micro-muscle movements, to discourage use of specific facial muscle groups, reduce or prevent wrinkles, and/or achieve a different repertoire of facial expressions, or for other self-improvement reasons.

### RELATED APPLICATIONS

The present application claims priority to U.S. Provisional Application No. 62/336,728, filed on May 15, 2016, entitled “Prevention of the Wrinkling and Expression of Human Skin” all of which are hereby incorporated by reference in its entirety for all purposes.

### FIELD OF THE DISCLOSURE

The present application generally relates to methods and systems for managing wrinkling, including but not limited to the use of a film that is attached to human skin.

### BACKGROUND

One reason a person develops and keeps frown lines, crow's feet, and other facial wrinkle patterns is because the person's repertoire of facial expressions includes recurring use of expressions, like brow furrowing, eyebrow raising, and others that repeatedly wrinkle the skin in specific ways.

An existing treatment for reducing signs of wrinkles is the use of neurotoxins like Botulinum toxin that aim to work by preventing specific muscle actions through which the offending wrinkles occur. The use of Botulinum toxin as a solution however suffers from numerous drawbacks, including cost, unpleasant subcutaneous delivery, unknown long-term neurotoxic effects, and a tendency to completely remove or render unnatural specific facial expressions from the user's repertoire for numbers of months at a time, due to the all-or-none action of the botulinum-induced paralysis. It would be beneficial therefore for an improved method for treating, preventing, minimizing, or diminishing signs of aging in the skin, in particular facial wrinkles.

### SUMMARY

In some aspects, the present disclosure relates to methods for treating, preventing, minimizing, and/or diminishing signs of aging in the skin, such as facial wrinkles. In some other aspects, the present disclosure relates to methods for training a user's face to favor some configurations (facial expressions) over others.

In some embodiments, the inventive concepts as described herein may be directed to a gel or other viscous liquid with specific properties that is applied to areas on the face or body, and may be configured to change state to a thin, adhesive semi-elastic transparent film that is left on human skin (e.g., on the face), over extended periods of time, for example 24-48 hours. In some embodiments, the specific properties of the gel as well as the method for its application on the skin are carefully configured in order to optimize the sensation (or force) feedback to the user in response to both the user's conscious and sub-conscious micro-muscle movements, for the purpose of discouraging the use of specific facial muscle groups, and to reduce or prevent wrinkles and/or achieving a different repertoire of facial expressions, or for other self-improvement reasons.

In one aspect, the present disclosure is directed to a method for managing wrinkling. The method may include applying a liquid or gel based substance on skin of a user. The liquid or gel based substance may form a film that adheres to the skin and having elasticity within the range of 0.02 newtons per millimeter (N/mm) to 0.2 N/mm. The film may have a limit for displacement that is greater than 4 mm. Displacement may correspond to a linear change in position of a reference point on an elastic material when force is applied, relative to an initial position of the reference point without application of force on the elastic material. Elasticity may correspond to force applied per unit displacement of the elastic material. The film may provide, to the user, force feedback that is at least 0.02 N when the displacement of a portion of the skin that is in contact with the film reaches 1 mm while the film remains adhered to the portion of the skin.

In some embodiments, the force feedback is designed or used to at least one of: indicate to the user that wrinkling of the skin is occurring, or prompt the user to limit or avoid the wrinkling. In certain embodiments, the method includes changing the one or more parameters of the film over at least a first portion of the film. In some embodiments, one or more parameters of the film may be changed by applying water or other fluid to the first portion of the film. In certain embodiments, changing the one or more parameters of the film includes changing one or more of: elasticity, adhesiveness, appearance, thickness, pore density or pore size of the film.

In certain embodiments, the film delivers one or more dermatropic agents to the skin. The film may be transparent, translucent or configured according to a color of the skin. The film may provide at least one of: breathability to the skin, a level of ultraviolet protection, an outer protective layer for the skin, a sheen compatible with that of the skin, a peel force of less than or equal to 0.8 N on the skin, or durability on the skin over at least a predefined period of time.

In some embodiments, applying the liquid or gel based substance on the skin of the user includes applying the liquid or gel based substance on a portion of the user's face or over a specific muscle or muscle group. The liquid or gel based substance may include at least one of: polyvinyl alcohol, propylene glycol or glycerin. In certain embodiments, a device is used to hold the skin in place as the film is being formed.

In another aspect, the present disclosure is directed to a product for managing wrinkling. The product may include a film formed from applying a liquid or gel based substance on skin of a user. The film may adhere to the skin and have elasticity within the range of 0.02 newtons per millimeter (N/mm) to 0.2 N/mm. The film may have a limit for displacement that is greater than 4 mm, wherein displacement may correspond to a linear change in position of a reference point on an elastic material when force is applied, relative to an initial position of the reference point without application of force on the elastic material. Elasticity may correspond to force applied per unit displacement of the elastic material. The file may provide force feedback to the user that is at least 0.02 N when the displacement of a portion of the skin that is in contact with the film reaches 1 mm while the film remains adhered to the portion of the skin.

In some embodiments, the film provides the force feedback to at least one of: indicate to the user that wrinkling of the skin is occurring, or prompt the user to limit or avoid the wrinkling. One or more parameters of a first portion of the film may be adjustable by applying water or other fluid to the first portion of the film. The one or more parameters of the film may include one or more of: elasticity, adhesiveness, appearance, thickness, pore density or pore size of the film. The film may deliver one or more dematropic agents to the skin. The film may be transparent, translucent, or have an appearance configured according to a color of the skin. The film may provide at least one of: breathability to the skin, a level of ultraviolet protection, an outer protective layer for the skin, a sheen compatible with that of the skin, a peel force of less than or equal to 0.8 N on the skin, or durability on the skin over at least a predefined period of time. In some embodiments, the film is formed on a portion of the user's face or over a specific muscle or muscle group. The liquid or gel based substance may include at least one of: polyvinyl alcohol, propylene glycol or glycerin.

### BRIEF DESCRIPTION OF THE FIGURES

The foregoing and other objects, aspects, features, and advantages of the present solution will become more apparent and better understood by referring to the following description taken in conjunction with the accompanying drawings, in which:

FIG. 1 depicts example placements of adhesive film relative to muscle groups;

FIG. 2 depicts example embodiments of adhesive film in operation when applied to skin;

FIG. 3 is an illustrative graphical representation of a function range of film;

FIG. 4 illustrates effects of different film elasticities on peel displacements and perceptible displacement thresholds; and

FIG. 5 is a flow diagram of an embodiment of a method for managing wrinkling.

The features and advantages of the present solution will become more apparent from the detailed description set forth below when taken in conjunction with the drawings, in which like reference characters identify corresponding elements throughout. In the drawings, like reference numbers generally indicate identical, functionally similar, and/or structurally similar elements.

### DETAILED DESCRIPTION

Some existing treatments aim to restrict facial movement and thereby retrain the user's expression repertoire to avoid specific wrinkle-causing movements. For example, adhesive-laden papers and tapes (e.g. Frownies™) are inelastic, resistant to both extension (e.g., stretching) and compression (e.g., high frequency folding). They are meant to adhere directly to wrinkling skin, with some cutting and/or other size/shape customization often needed to tailor the tape to the shape of individual facial regions, and they do prevent the skin under the tape from wrinkling into its existing patterns. However, unlike botulinum, they do not paralyze but merely impede muscle action, as will be described in more detail below. The result is typically facial expressions of diminished amplitude, but with an unnatural buckling and/or low-frequency folding on the inelastic tape. The tape itself is also very visible, especially at the edges between taped and un-taped areas.

These visibility issues are not typically a problem for the tape's intended usage, i.e., as a private, e.g., night-time application, and the tape itself is not designed for invisibility or even subtleness. However, an additional problem with such tape, with respect to both functionality and visibility, is the combination of inelasticity and the requirement for a comfortably low maximum force required to peel off the tape, as this means that normal forces of facial muscle activity can cause peeling to start. For example, a typical measured peel-off force, for a comfortable low adhesion bandage material such 3M Durapore, as measured in laboratory studies is approximately 0.8 Newton per 25.4 mm wide adhesive strip, as the force reaches a steady state for a 400 millimeter per minute (mm/min) constant velocity displacement at 180 degrees.

The present solution, in some embodiments, is based primarily on four quantitative factors: a measure of the maximum muscle-induced facial displacement, a measure of perceptible displacement threshold, a measure of the comfortable peel force, and a measure of the smallest perceptible sheer force, as will be discussed later. In some embodiments, the method for managing wrinkling comprises applying a liquid or gel based substance on skin of a user, forming, by the liquid or gel based substance, a film that adheres to the skin and having elasticity within a particular range, with a preferred range being between the range of 0.02 newtons per millimeter (N/mm) to 0.2 N/mm, and above a minimum threshold for displacement, with a preferred threshold being 4mm. Experiments were performed to measure these factors, and the findings are presented in Table 1 below and used in a particular embodiment of the present solution. In related embodiments, the method may comprise applying the liquid or gel based substance on the skin of the user on a portion of the user's face or over a specific muscle or muscle group.

In some embodiments, the force feedback is configured to indicate to the user that wrinkling of the skin is occurring, or prompt the user to limit or avoid the wrinkling. For example, the skin itself contains force feedback receptors, as described below in more detail, that can provide such a force feedback mechanism directly to the human nervous system to provide a pressure sensation to the user, but only if a film with suitable mechanical properties, as described below, is applied to the skin.

In some embodiments, the present solution may include means other than film to sense that the user is wrinkling the face, and upon sensing wrinkling, a means to provide feedback to the user that wrinkling is occurring. In some embodiments, the method of sensing may be a video camera acquiring video and/or tracking/analyzing images of a user's face, and the method of providing feedback may be presenting the video to the user on a screen, such as on a mobile phone, or by providing tactile feedback (e.g., vibration to a wristband or to a phone) coded to particular expressions.

A force comfortably achievable by facial muscles, for example the occipitofrontalis, is approximately 8 Newtons per 25.4 mm, as measured in our research, in which subjects attempted to lift their eyebrows while weights of different loads had been taped to a brow with a 25.4×10 mm strip of high-adhesive tape. The fact that a lateral force achievable by the face is an order of magnitude above a typical peel force of tape (8 instead of 0.8 Newtons) means that for an inelastic tape like Frownies™, the tape will likely peel with increasing facial activity, such as would likely occur during typical day-time usage.

TABLE 1
Measures and values cited in this document

| | |
|---|---|
| Measurement | Value |

Force Measurements

| | |
|---|---|
| comfortable peel force | 0.8 Newtons |
| smallest perceptible force | 0.02 Newtons |

Displacement Measurements

| | |
|---|---|
| maximum skin stretch | 4.0 millimeters |
| perceptible displacement threshold | 1.0 millimeters |

Psychophysical Constants

| | |
|---|---|
| Weber Fraction for tactile sensation | 0.20 |

As a treatment for wrinkle reduction, a liquid may be applied to the wrinkled areas of the face, and this liquid then forms an inelastic adhesive film. For example, sodium polystyrene sulfonate can be applied to the face as a liquid and shrinks as it forms an adhesive film; and like Frownies™, it is meant to be applied directly to wrinkled areas, and after film formation, is meant to perform a similar immobilizing function on the wrinkles themselves. Such a film is also similar to Frownies™ and other adhesive tapes in that it has no measurable elasticity, and is in fact worse than these tapes with respect to edge lifting and peeling, because (a) unlike tape, it is brittle to any bending or shear forces, and (b) the approximately five percent shrinkage per linear dimension, as measured by our research, produces upon the face a constant shear force of approximately one Newton per 25.4 mm that, even without any voluntary facial movement, causes noticeable pulling, peeling and cracking at the edges of the applied film.

Although these tapes and other films do provide some benefit based on their ability to impede specific facial muscle groups and thereby to retrain the face to refrain from performing specific wrinkle-causing expressions, their utility is limited by their visibility. That is, with more hours per day of usage there is more opportunity for facial training, but to take advantage of this opportunity, users may require a more discreet, preferably invisible treatment that can withstand typical day-time activities.

One visibility-causing problem that such tape and liquid treatments share is based on their inelasticity. Although inelasticity of materials does provide better immobilization of the face than can be provided by more elastic materials, it also causes visibility problems such as the buckling and peeling described above.

In some embodiments, these issues are addressed by changing one or more parameters of the film over at least a first portion of the film. In some embodiments, our approach is based on our finding that, using modern skin-safe components such as poly-vinyl alcohol, and known synthesis techniques to modify the elasticity constant of these materials, we can, through a novel technique to be described below, optimize this constant to a range resulting in films that are much more elastic (e.g., more pliable, with a lower Hooke's Constant) than other tapes or films, providing a more subtle sensory feedback signal than the full immobilization provided by inelastic materials, and that this more subtle sensory feedback signal provides sufficient feedback for facial expression retraining, doing so in a way that, unlike inelastic materials, provides a means for long-term, invisible (e.g., less visually discernable or apparent) day-time usage.

In some embodiments, the present solution may be a gel or other viscous liquid configured to have specific properties that is applied to areas of the face surrounding brow-furrowing wrinkles, frowning wrinkles, and/or any other facial region that naturally stretches in conjunction with facial actions that the user would like to minimize, and which then changes state to a thin (e.g., low profile, at least in part conforming to the profile of the underlying skin), adhesive, transparent, semi-elastic film structure that provides specific force feedback to the user that is optimal in the minimization of wrinkling. The gel with these specific properties may be referred as a “structure gel” to capture the effect of starting as a gel and changing state to a film structure with specific properties and mechanisms of operation as described below. In some embodiments, the film may be configured to be transparent, translucent or of a certain appearance.

In some embodiments, we have determined a method for optimizing the elasticity constant of the film material such that the elasticity constant is greater than the elasticity constant required to provide a minimum perceptible stretch force sufficient for facial expression training, and less than the elasticity constant that results in a peel force that results in peeling, cracking or tearing of the film, or results in any other effect that results in visibility of the film, or change (e.g., physical distortion) in the film over a time period.

In some embodiments, a second aspect of our approach concerns the form of this more subtle sensory feedback signal, which differs in kind from that of the alternative treatments, as follows. For these alternative treatments, which are meant to be applied directly onto the wrinkled regions and to adhere fully and without any elastic “give”, immobilization is generally effective; that is, users have difficulty applying sufficient muscle force to buckle the tape and draw the face into the intended expression, e.g., brow raising. For this reason, the sensory effect is largely proprioceptive in nature; that is, users experience primarily the muscular effort of trying to move the face, rather than, for example, any change in tactile sensation of the tape or film on the skin of the face. During intended facial movements, tactile sensations, such as lateral pulling of the skin at the edges of the tape and lifting and pressure on the skin under the hills and valleys of the buckling regions, can be recognized by users, but these are not reported as salient compared to the force feedback from the full immobilization.

In contrast to these existing approaches, in some embodiments, the present solution does not use immobilization, and instead relies primarily on the pulling sensations evoked on the skin surface by the resistance of our more elastic film to intended facial movements. Although these pulling sensations are more subtle than the muscle-sensing based feedback in the other treatments, our studies have shown no measurable difference in training effectiveness, and, unlike the other treatments, no measurable peeling or cracking even after a full day's usage. Put another way, in some embodiments, we have determined a method for optimizing the elasticity constant of the film material such that the elasticity constant is greater than the elasticity constant required to provide a minimum perceptible stretch force sufficient for facial expression training, and less than the elasticity constant that results in immobilization.

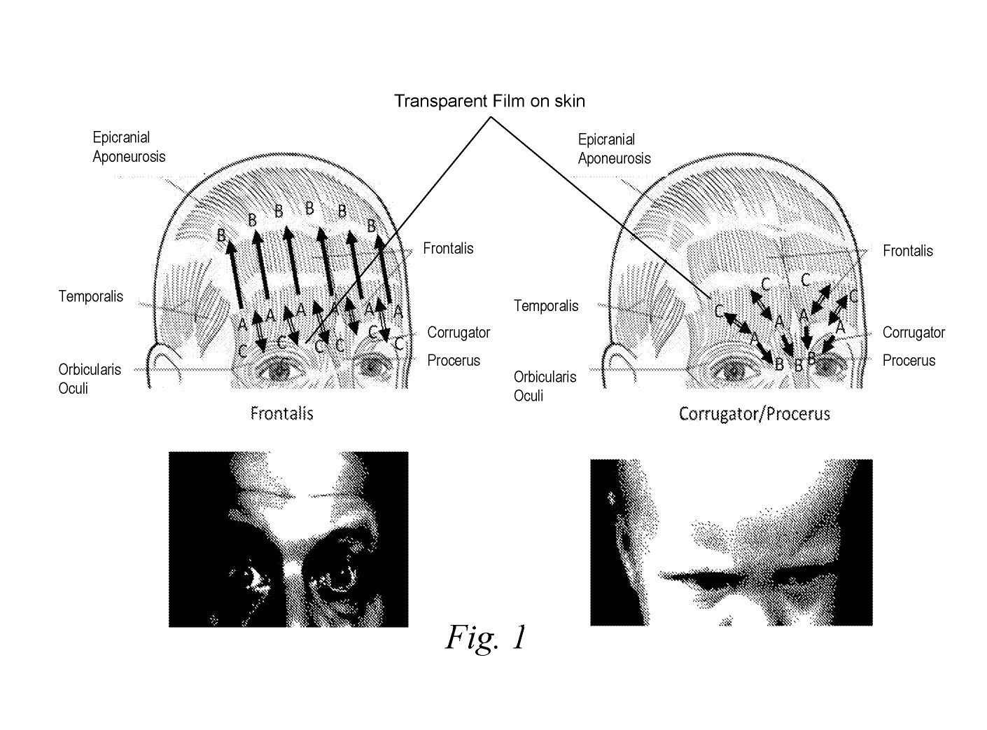

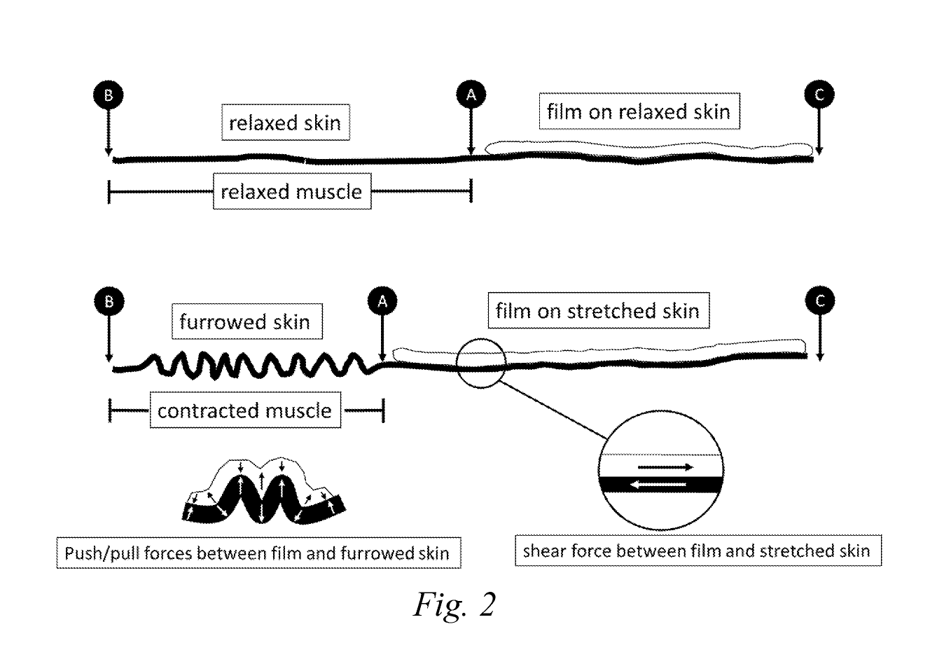

Furthermore, although the fully immobilizing tape or film of the alternative approaches is intended to be applied directly to a wrinkled region, another difference resulting from our reliance on stretch sensitivity is that our film is intended to be applied to a different region that includes the region surrounding the wrinkled region, for reasons that can be clarified using FIGS. 1 and 2.

Referring to FIG. 1, insertion (A) and origin (B) points of two different muscle groups, along with direction of motion during muscle activation (solid arrows), and accompanying skin stretch region (open arrows) ending as indicated by labels C, are depicted. Resulting facial expressions for the two different muscles groups are shown in the images at bottom, with characteristic horizontal furrowing shown in the face to the left. For the image to the right, the characteristic vertical furrows caused by corrugator and procerus use are not visible in this child subject. One or more films can be applied or attached to one or more of the muscle groups, for instance transparently over and/or along certain of the identified direction(s) and region(s).

In these figures, points A and B may indicate the insertion and origin points respectively of a facial muscle. For example, for the occipito-frontalis muscles in the image to the left of FIG. 1, the origin is at a fixed point at the top of the head (the epicranial aponeurosis) and the insertion is onto the skin along the tops of the eyebrows and the top of the bridge of the nose. When these muscles are activated, the eyebrows go up and the skin of the forehead typically furrows into horizontal wrinkles, indicated in FIG. 2 in cross-section by the “furrowed skin” labeled region on the bottom panel of the figure, between points A and B. What is less apparent but at the same time, a physically necessary concomitant of this wrinkling skin is a region of stretching skin, to the other side of the insertion point; that is, between points A and C in both figures. In the case of the occipito-frontalis muscle to the left in FIG. 1, this stretching region is along the eyebrow ridges, through the bridge of the nose. As indicated in the right-hand image of FIG. 1, for the corrugator and procerus muscles—that typically produce a vertical line wrinkling region between the eyes—the stretching region extends from the middle of the top of each eyebrow, diagonally upward toward each adjacent upper temple.

In some embodiments, the film is configured to provide at least one of: breathability to the skin, a level of ultraviolet protection, an outer protective layer for the skin, a sheen or texture compatible with that of the skin, a peel force of less than or equal to 0.8 N on the skin, or durability on the skin over at least a predefined period of time.

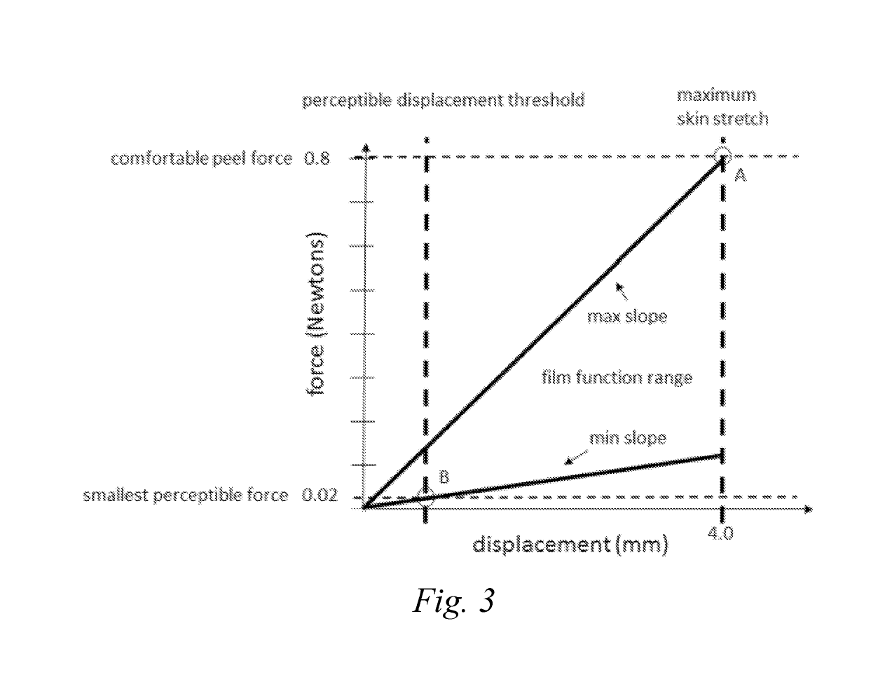

FIG. 2 provides a demonstration of adhesive film in operation when applied to skin, and can show the mechanism of the film when applied to stretching and furrowing regions of the skin, such as those labeled in FIG. 1. For example shear forces can result from application of adhesive film to stretching skin located away from a furrowed region, as compared push/pull forces from application of adhesive film to a furrowed region. For the case of stretching, when the skin is stretched to a given linear displacement along a direction such as those indicated in FIG. 1, the attached film may be stretched along with it, and can cause a shear force in the opposite direction, dependent on the elasticity properties of the film, as measurable for example by a stress/strain function relating Newtons of resisting force to mm of linear displacement. For example, FIG. 3, which graphically presents an example method for determining the optimal such elasticity function, shows two such stress/strain functions, labeled “max slope” and “min slope”. These indicate the elasticity slope range of the optimized film, as will be discussed shortly.

For our purposes, stress/strain measurements to characterize a film can and should be done on the isolated film; i.e., not adhering to the skin, and show the measured spring force of the film as a function of displacement. For these films and displacements under consideration, the drawn linear function is a good approximation, and can therefore be characterized by a single slope parameter, called Hooke's Constant.

FIG. 2 also shows, in the bottom left diagram, the resulting forces when the film is applied to the furrowing regions, as opposed to the stretching regions of the skin. Although this effect does contribute somewhat to the overall tactile feedback percept introduced above, in our studies this feedback is dominated by the stretch-based tactile sensation, quantification of which contributes directly to our method for determining optimal film elasticity. This method can now be discussed in detail, with reference to FIG. 3, which illustrates how optimal film elasticity may be determined based on desired comfortable peel force and minimum perceptible stretch force.

The axes of the plot in FIG. 3 are, on the abscissa, displacement (in millimeters) and on the ordinate force (in Newtons). Onto this plot we place a thin horizontal dashed lines indicating the comfortable peel force, estimated here as 0.8 Newtons from our tests. We also place a lower horizontal dashed line at the smallest perceptible shear force, which for example is determined by applying a 25.4×10 mm adhesive strip on the facial skin area of interest (in our case, above the brow), and then measuring the minimum lateral displacement force that is reliably noticed by the subject. This perception threshold is estimated here as 0.02 Newtons.

To these two force measures on the ordinate, we can add two displacement measures onto the abscissa. One of these is the maximum muscle-induced facial displacement. That is, for a given maximum force measurement, e.g., the eyebrow raising measure discussed above, we also estimate the resulting skin stretch. For our example here, this measured maximum displacement is 4 mm, as shown by the vertical heavy dashed line to the right of the plot.

The other displacement measure is the perceptible displacement threshold. This can be determined as the displacement beyond which furrows are likely to occur, and therefore represents the displacement by which we want to be sure that sensory feedback has begun. In our research, this value can be measured or estimated as one fourth of the maximum of 4.0 mm, giving 1.0 mm.

Given these four measures, the optimal film elasticity may be determined, in summary, by finding a Hooke's Constant that is (a) sufficiently low so that full force facial expressions do not cause pulling on the film beyond an established comfortable peel force, yet (b) sufficiently high so that the tactile feedback may be felt before skin displacement has progressed to the point of furrow formation.

In reference to FIG. 3, in one embodiment a specific method of finding this optimum can be enumerated as follows:
Find the intersection of the horizontal “comfortable peel force” line with the vertical “maximum skin stretch” line (point A). A straight line drawn from the origin through this point can represent the elasticity function with the maximum allowable slope, which can ensure that local shear forces will not be so high as to cause peeling under normal usage.
Find the intersection of the horizontal “smallest perceptible force” line with the vertical “perceptible displacement threshold” line (point B). A straight line drawn from the origin through this point can represent the elasticity function with the minimum allowable slope, which can ensure that sensory feedback occurs before a furrow-inducing displacement can occur.

The triangular area in this plot, labeled “film function range”, can represent the range over which film elasticity functions may be safely chosen according to the criteria described here, for the assumed set of values on each of the four parameters. Within this range, different functions may be chosen based on manufacturing considerations, or on individual differences in the measurements (e.g., between individuals or between groups such as young vs old), or on personal preferences, such as for different perceptible displacement thresholds to modify expressiveness vs furrowing likelihood. In any case, our ability to synthesize films with different elasticity parameters means that we can produce a wide variety of such film variants to meet different population and individual needs.

In other words, in some embodiments, our method can optimize the elasticity constant of the film material as a function of four quantitative factors: a measure of the maximum muscle-induced facial displacement, a measure of perceptible displacement threshold, a measure of the comfortable peel force, and a measure of the smallest perceptible sheer force. In some embodiments, the elasticity of the film material may be less than the ratio of the comfortable peel factor force, and the maximum muscle-induced facial displacement. In some embodiments, the elasticity of the film material may be greater than the ratio of smallest perceptible sheer force, and the perceptible displacement threshold.

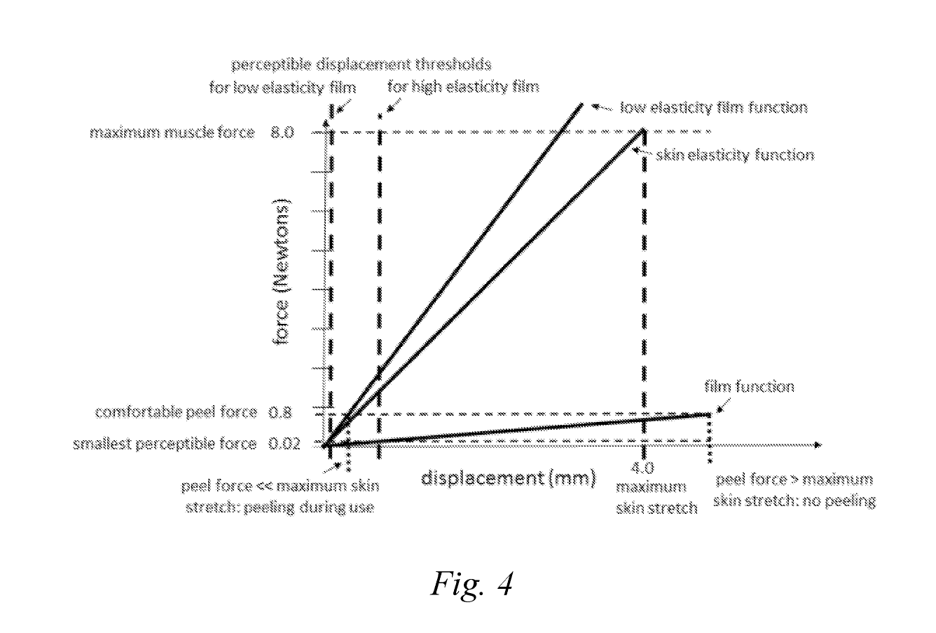

Referring now to FIG. 4, effects of different film elasticities on peel displacements and perceptible displacement thresholds are illustrated. Using the same plotting conventions as FIG. 3 but with a broader range of forces, FIG. 4 compares the effect of the elasticities in our method with those of alternative tape and film treatments. As a frame of reference for this analysis, we start with an estimated elasticity function for skin itself, which we estimate as a straight line through the origin and defined by the skin displacement corresponding to the maximum muscle force, as shown in the figure by the line labeled “skin elasticity function”. The film function range from FIG. 3 is replaced in FIG. 4 by a single estimated optimal film function, and appears with a much lower slope here, due to the wider ordinate range in FIG. 4. Also shown for comparison purposes in FIG. 4 is an elasticity function labeled “low elasticity film function”, with a slope higher than that of skin. This function is meant to represent a more moderate version of functions for existing tape and film treatments which, as discussed above, have no functional elasticity.

Even with this more moderate version of a low elasticity treatment, however, two interesting differences between its performance and that of our more elastic function (labeled “film function” in the figure) emerge. First, if we look at the intersection of each function with the peel force line, we see that while for our high elasticity film this intersection is, as designed, at a displacement above that achievable by intended facial movements alone (as indicated by the vertical thick dotted lined labeled “peel force>maximum skin stretch”), for the alternative treatments this intersection is at a much lower displacement, less than one eighth the excursion from resting facial position to full displacement, at a point indicated by the vertical dotted line labeled “peel force<<maximum skin stretch”. This gives a dramatic graphical explanation for the peeling that is observed in practice with these other treatments.

Second, if we look at the intersection of each of these two functions with the horizontal “smallest perceptible force” line, we observe that the resulting perceptible displacement threshold is near zero for the low elasticity treatment, meaning that any facial displacement at all can cause a force feedback, and therefore even minimal expressions can be constrained. In contrast, for our high elasticity film, as described above, the perceptible displacement threshold is at about one quarter full displacement, a value that permits some facial expressiveness but guards against excessive motion and its resultant furrowing. Put in other words, in some embodiments, the elasticity of the film is chosen such that the perceptible displacement threshold is greater than a threshold. In some embodiments, the elasticity of the film is chosen such that the perceptible displacement threshold is greater than one quarter of the maximum muscle-induced displacement.

One other instructive difference between our treatment and any treatment that shrinks (like the sodium polystyrene sulfonate film described above) is as follows. In addition to the latter's deleterious effects in causing peeling and cracking, the constant shearing force caused by this shrinkage can also reduce sensitivity to the sensory feedback signal described above when facial movement is occurring since, as taught by Weber's Law, the perceptual magnitude of the change in a sensor signal is proportional to the base level of that signal. To determine magnitude of this desensitization, we can use estimates of the Weber fraction of tactile sensation. Here, a typical Weber fraction may be 0.20, meaning in this context that the additional force required to detect a change in stretch can be approximately 20% of the base force of 1 Newton estimated above, or 0.2 Newtons. This can be compared with the absolute sensitivity of facial skin to shear forces; i.e., without any base force, estimated as described above to be approximately 0.02 Newtons. As the threshold of 0.2 Newtons for the pre-stretched skin is an order of magnitude above 0.02 Newtons for the threshold from non-stretched skin, we conclude that this large starting shear can significantly reduce the efficacy of a shrinking film, like sodium polystyrene sulfonate, in providing sensory feedback for dissuading specific facial movements. With respect to the plotting construction of FIG. 3, this increase in threshold can represent an increase in the plotted smallest perceptible force, and therefore an increase in the min slope.

For the case of the sodium polystyrene sulfonate film described above, the min slope increase can be so great as to be larger than the allowable max slope, thereby rendering the film unusable according to these design criteria. However, for initial shrinkage based stretch forces significantly lower than the 1 Newton force described in this example, in some embodiments of the present solution, a small amount of shrinkage, for example with an initial force of 0.05 Newtons, is formulated. For small amounts like this and others, the min slope to max slope range can be formulated to be positive, and some advantages can accrue. For example, this initial shrinkage can have the function, in effect, of taking up some initial slack in the skin, so that sensitivity to the feedback signal can begin to have an effect earlier in the displacement trajectory of the face. This initial sensitization at the start of a Weber's Law based masking function is analogous to that in sine grating contrast sensitivity in vision.

In some embodiments, the present solution has further advantages beyond preventing wrinkling. These include for example the ability of the structure gel to include healthful dermatropic skin agents, such as humectants, exfoliants and vitamins that can be delivered to the skin through the gel, and then be protected by the developing film above. It can also include various other protective agents, such as UV filters.

In some embodiments, emulsified components that move more slowly into the skin can also be included, for the purpose of providing a more realistic, microscopically slightly roughened surface that avoids the sheen of some films. These emulsified components can also be designed to leave behind pores in the film, for breathability and additional naturalness.

In some embodiments, one or more parameters of the film are adjusted by, for example, the application of water or other fluid to the film. Also in certain embodiments, other parameters of the film that may be adjusted include one or more of: elasticity, adhesiveness, appearance, thickness, pore density or pore size of the film. In some embodiments, the liquid or gel based substance comprises at least one of: polyvinyl alcohol, propylene glycol or glycerin. In one particular embodiment of the structure gel, the basic structure of the film is provided by polyvinyl alcohol, which has been demonstrated to be safe for application on human skin through numerous widespread existing facial treatments (e.g., peel-off skin masks designed to remove impurities), as well as contact lenses and clothing. Moreover, various physical parameters of polyvinyl alcohol based films, including elasticity, drying time, adhesiveness, and others, have been shown to be readily modifiable by varying the quantities of other ingredients in the formulation, such as water, glycerin, polyethylene glycol, propylene glycol, SD alcohol and others, and by preparation modifications such as inserting different numbers of freeze/thaw cycles. With these and other methods, properties relevant to the formulation's use as a long-lasting, invisible, and/or semi-elastic film in the current invention can be easily optimized for different user populations and purposes.

In another embodiment, different sets of physical parameters can be utilized within different portions of the applied film, so that the properties of the film can be optimized locally or non-uniformly across different regions of the face or other attached skin. For example, adding more water, either for a separate sample of the gel, or after the gel has been applied to a specific region of the face, can create a thinner film, which can be more easily feathered into bare skin to minimize visibility of the edges. Similarly, higher polyvinyl alcohol concentration and/or other manipulations can create a more adherent film, for facial regions with higher displacements and thus more risk of accidental peeling. Additionally, changing the concentrations of glycerin and propylene glycol and/or other ingredients can result in films with different elasticity properties, so that different amounts of perceptual feedback across different facial regions can be achieved, for example for customizing the range and types of facial expressions that need to be especially discouraged to minimize wrinkling in a specific individual. Different elasticities are also useful in films applied directly onto wrinkles versus those applied to stretching areas of the skin, such as are illustrated in FIG. 2.

In some embodiments, the film includes one or more mechanisms for delivering to the skin one or more dermatropic agents, such as moisturizing elements. These can, for example, be included as emulsified components that are readily absorbed by the skin as the film is drying.

In other embodiments, different pore sizes can be created and/or maintained in the film, based for example on voids left in the film by the skin absorption of the emulsified components. These pore sizes can be customized to the individual and/or across different regions of the face, as described above. This may be useful, for example, to maintain breathability of the skin, and to customize it, based for example on individual or local differences in sweat response and therefore of the need to transfer sweat from the interior to the exterior of the film.

In another embodiment, the glossiness and/or texture of the film can be modified, for example to remove any film sheen that may be objectionable to some users. One way to achieve this is to modify the pore density and/or size, using the same emulsification mechanism described above.

In another embodiment, the film's transparency, translucency and/or color can be modified according to the color of the skin, and/or the preferences of the user for skin tone modification, based on the addition of skin-safe dyes such as used in ordinary makeup.

In some embodiments, it is useful to provide a means to immobilize the face while the gel is setting, drying or otherwise transitioning into its film form. For example, this prevents a habitually frowning user from frowning during this setting process, which would have the deleterious effect of setting the film in a manner that would tend to provide perceptual feedback to maintain the frown, as well as setting the skin into an already wrinkled state. One method for immobilization uses a headband like device, but with one contact surface across the hairline, another contact surface across the eyebrows, and a mechanical means to maintain a non-frowning separation between these two facial regions. This immobilization device makes it difficult to frown while the gel is setting, and the device is typically removed after the film has completely set.

In some embodiments an additional benefit of the structure gel is that the resulting film can be used to modify other facial motions, beyond the expressions such as brow-furrowing, squinting and lip-pursing that are responsible for some wrinkles. For example, in some embodiments, the present solution is a practical low-cost method to provide feedback against facial tics and grimaces. And in individuals for whom wrinkle reduction is not a goal, the structure gel may still be used by individuals who desire help in modifying their own repertoire of facial expressions, for example to reduce perceived excess frowning, lip-pursing, squinting, single eyebrow raising, etc.

The present solution provides a low cost, safe, painless, easy to apply treatment that provides wrinkle reduction/expression retraining without the unnatural appearance, potential danger, or cost of botulinum paralysis, the visibility and inconvenience of tape, or the impermanence, visibility and sub-optimal feedback of available film-forming liquids.

As a thin, transparent film optimized for long-term breathability, adherence and elasticity, the treatment is not readily detectable to others, and can therefore be left on the face for long periods of time, for these long-lasting beneficial effects and others, and then peeled off when desired.

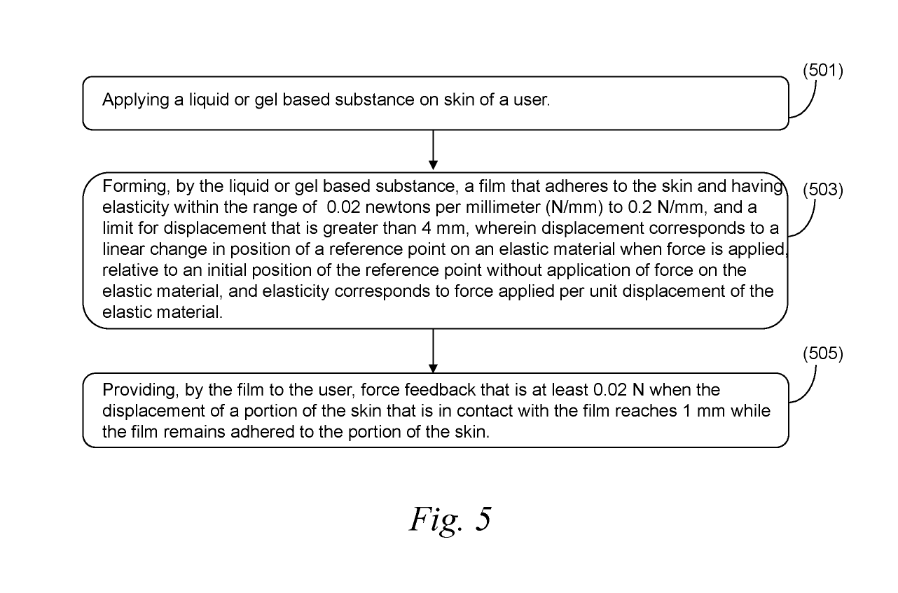

Referring to FIG. 5, one embodiment of a method for managing wrinkling is depicted. The method may include applying a liquid or gel based substance on skin of a user (501). The method may include forming, by the liquid or gel based substance, a film that adheres to the skin and having elasticity within the range of 0.02 N/mm to 0.2 N/mm (503). The film may have a limit for displacement that is greater than 4 mm. Displacement may correspond to a linear change in position of a reference point on an elastic material when force is applied, relative to an initial position of the reference point without application of force on the elastic material. Elasticity may correspond to force applied per unit displacement of the elastic material. The film may provide, to the user, force feedback that is at least 0.02 N when the displacement of a portion of the skin that is in contact with the film reaches 1 mm while the film remains adhered to the portion of the skin (505).

Referring to (501), and in some embodiments, a liquid or gel based substance may be applied on skin of a user. The skin may be prepared prior to application of the substance, for example cleansed, dried or moistened, shaved of hair, and/or applied with another substance to improve adhesiveness to the skin. A user may apply the substance using a device or tool, such as a spreader and/or a syringe or other dispenser. A component or additive of the substance may be applied separately, using a similar or different device or tool, such as spraying device. The skin being applied with the substance may be maintained in a defined orientation or position over a certain period of time. For example, a main portion of the skin may be kept primarily or substantially horizontal and/or facing upwards, or vertical and/or facing to one side, as the substance is applied. In some embodiments, the skin may be matched or coupled to a mold or application guide, to distribute the substance evenly or in a particular fashion over the skin.

In some embodiments, the liquid or gel based substance is applied on a portion of the user's face or over a specific muscle or muscle group. The liquid or gel based substance may be applied along a certain direction (e.g., corresponding to a direction of motion of skin/muscle due to muscle activation) and/or relative to certain wrinkles or frown line(s). The substance may be applied in any quantity, over one or more areas of the skin. The substance may be applied in layers, in some embodiments.

A fan or blower may be used to apply the substance, e.g., by spreading the substance more uniformly or in a certain configuration. In some embodiments, the substance is applied in a semi-solidified form as a strip or layer, e.g., which may be cut or sliced to a desired shape prior to application. The liquid or gel based substance may include polyvinyl alcohol, propylene glycol, glycerin and/or other substances or compounds.

Referring to (503), and in some embodiments, a film that adheres to the skin may be formed by the liquid or gel based substance. In certain embodiments, a device holds the skin in place as the film is being formed. The film may be formed while using a mask, band or other device to support or hold at least some portion(s) of the skin in place (e.g., in a non-furrowed or stretched state, in a certain position, or otherwise). Some amount of air, heat and/or radiation may be applied to the substance to help form the film, e.g., by speeding up the process. The substance may be allowed to dry or set on the skin over a certain period of time (e.g., 10 mins, depending on the amount of the substance used).

The file may have elasticity within the range of 0.02 N/mm to 0.2 N/mm (503). The film may have a limit for displacement that is greater than 4 mm. Displacement may correspond to a linear change in position of a reference point on an elastic material when force is applied, relative to an initial position of the reference point without application of force on the elastic material. Elasticity may correspond to force applied per unit displacement of the elastic material.

In certain embodiments, the film delivers one or more dermatropic agents to the skin. The film may be transparent, translucent or configured according to a color of the skin. The film may provide at least one of: breathability to the skin, a level of ultraviolet protection, an outer protective layer for the skin, a sheen compatible with that of the skin, a peel force of less than or equal to 0.8 N on the skin, or durability on the skin over at least a predefined period of time.

In certain embodiments, the method includes changing the one or more parameters of the film over at least a first portion of the film. The change can be made during the forming of the film, or after the film has formed. In some embodiments, one or more parameters of the film may be changed by applying water or other fluid to the first portion of the film. In certain embodiments, changing the one or more parameters of the film includes changing one or more of: elasticity, adhesiveness, appearance, thickness, surface texture, pore density or pore size of the film. Some portions of the substance can be massaged or molded onto various skin portions and profiles, e.g., before they are set into film form, or via adding water or other fluid after the film is formed.

Referring to (505), and in some embodiments, the film may provide, to the user, force feedback that is at least 0.02 N when the displacement of a portion of the skin that is in contact with the film reaches 1 mm while the film remains adhered to the portion of the skin. The elasticity or tension of the film enables the user to feel certain displacements of the skin. In some embodiments, the force feedback is designed or used to at least one of: indicate to the user that wrinkling of the skin is occurring, or prompt the user to limit or avoid the wrinkling.

While various embodiments of the methods, products and systems have been described, these embodiments are exemplary and in no way limit the scope of the described methods, products or systems. Those having skill in the relevant art can effect changes to form and details of the described methods, products and systems without departing from the broadest scope of the described methods, products and systems. Thus, the scope of the methods, products and systems described herein should not be limited by any of the exemplary embodiments and should be defined in accordance with the accompanying claims and their equivalents.

We claim:

1. A method for managing wrinkling, the method comprising:
applying a liquid or gel based substance on skin of a user;
forming, by the liquid or gel based substance, a film that adheres to the skin and having elasticity within the range of 0.02 newtons per millimeter (N/mm) to 0.2 N/mm, and a limit for displacement that is greater than 4 mm, wherein displacement corresponds to a linear change in position of a reference point on an elastic material when force is applied, relative to an initial position of the reference point without application of force on the elastic material, and elasticity corresponds to force applied per unit displacement of the elastic material; and
providing, by the film to the user, force feedback that is at least 0.02 N when the displacement of a portion of the skin that is in contact with the film reaches 1 mm while the film remains adhered to the portion of the skin.

2. The method of claim 1, wherein the force feedback is configured to at least one of: indicate to the user that wrinkling of the skin is occurring, or prompt the user to limit or avoid the wrinkling.

3. The method of claim 1, further comprising changing the one or more parameters of the film over at least a first portion of the film.

4. The method of claim 3, wherein changing the one or more parameters of the film comprises applying water or other fluid to the first portion of the film.

5. The method of claim 3, wherein changing the one or more parameters of the film comprises changing one or more of: elasticity, adhesiveness, appearance, thickness, pore density or pore size of the film.

6. The method of claim 1, further comprising delivering, by the film to the skin, one or more dematropic agents.

7. The method of claim 1, wherein the film is transparent, translucent or configured according to a color of the skin.

8. The method of claim 1, wherein the film is configured to provide at least one of: breathability to the skin, a level of ultraviolet protection, an outer protective layer for the skin, a sheen compatible with that of the skin, a peel force of less than or equal to 0.8 N on the skin, or durability on the skin over at least a predefined period of time.

9. The method of claim 1, wherein applying the liquid or gel based substance on the skin of the user comprises applying the liquid or gel based substance on a portion of the user's face or over a specific muscle or muscle group.

10. The method of claim 1, wherein the liquid or gel based substance comprises at least one of: polyvinyl alcohol, propylene glycol or glycerin.

11. The method of claim 1, wherein forming the film comprises using a device to hold the skin in place as the film is being formed.

12. A product for managing wrinkling, the product comprising:
a film formed from applying a liquid or gel based substance on skin of a user, the film configured to:
adhere to the skin and have elasticity within the range of 0.02 newtons per millimeter (N/mm) to 0.2 N/mm, and a limit for displacement that is greater than 4 mm, wherein displacement corresponds to a linear change in position of a reference point on an elastic material when force is applied, relative to an initial position of the reference point without application of force on the elastic material, and elasticity corresponds to force applied per unit displacement of the elastic material; and
provide force feedback to the user that is at least 0.02 N when the displacement of a portion of the skin that is in contact with the film reaches 1 mm while the film remains adhered to the portion of the skin.

13. The product of claim 12, wherein the film is configured to provide the force feedback to at least one of: indicate to the user that wrinkling of the skin is occurring, or prompt the user to limit or avoid the wrinkling.

14. The product of claim 12, wherein one or more parameters of a first portion of the film is adjustable by applying water or other fluid to the first portion of the film.

15. The product of claim 14, wherein the one or more parameters of the film comprises one or more of: elasticity, adhesiveness, appearance, thickness, pore density or pore size of the film.

16. The product of claim 12, wherein the film is further configured to deliver one or more dematropic agents to the skin.

17. The product of claim 12, wherein the film is configured to be transparent, to be translucent, or to have an appearance configured for the user.

18. The product of claim 12, wherein the film is configured to provide at least one of:
breathability to the skin, a level of ultraviolet protection, an outer protective layer for the skin, a sheen compatible with that of the skin, a peel force of less than or equal to 0.8 N on the skin, or durability on the skin over at least a predefined period of time.

19. The product of claim 12, wherein the film is further configured to be formed on a portion of the user's face or over a specific muscle or muscle group.

20. The product of claim 12, wherein the liquid or gel based substance comprises at least one of: polyvinyl alcohol, propylene glycol or glycerin.
